(19) United States
(12) Patent Application Publication
(10) Pub. No.: US 2026/0355090 A1
(43) Pub. Date: Nov. 12, 2026

(54) TOOTH WHITENING COMPOSITION WITH STAGED SYNERGISTIC RELEASE AND PREPARATION METHOD THEREOF

(71) Applicant: Jiangxi Dentalbright Technology Co., Ltd, Jiujiang (CN)

(72) Inventors: Junyuan Qin, Nanchang (CN); Wei Min, Nanchang (CN); Yingying Dai, Nanyang (CN)

(21) Appl. No.: 19/708,029

(22) Filed: Jun. 15, 2026

(30) Foreign Application Priority Data
May 20, 2026 (CN) 202610701350.3

(51) Int. Cl. A61K 8/66; A61Q 11/00

(52) U.S. Cl. CPC A61K 8/66; A61Q 11/00; A61K 2800/28

(57) ABSTRACT

A tooth whitening composition with staged synergistic release and a preparation method are provided. The composition includes enzymatic film degradation component, chelating desorption component, porous adsorption material, enamel repairing material, abrasive base, first porous carrier (pore size 50-100 nm), second porous carrier (pore size 2-20 nm), surfactant, sweetener and flavor. The enzymatic film degradation component is loaded on the first porous carrier, and the chelating desorption component is loaded on the second porous carrier. Three-stage release is realized by pore size difference: in the first stage (0-1 min), the enzymatic component rapidly decomposes acquired pellicle; in the second stage (2-3 min), the chelating component cleaves the stain binding bond; and in the third stage (after 2 min), the adsorption material and the repairing material absorb the stain and repair the enamel.

### CROSS-REFERENCE TO RELATED APPLICATIONS

This application claims priority to Chinese Patent Application No. 202610701350.3, filed on May 20, 2026, which is hereby incorporated by reference in its entirety.

### TECHNICAL FIELD

The present disclosure relates to the technical field of oral care, and in particular to a tooth whitening composition with staged synergistic release function and a preparation method thereof.

### BACKGROUND

With the improvement of people's living standards and the enhancement of aesthetic consciousness, tooth whitening has become an important development direction in the field of oral care. Coffee, tea, red wine and tobacco in daily diet are easy to form extrinsic stain deposits on the surface of teeth, which leads to yellowing or darkening of teeth and affects aesthetics.

Studies have shown that the formation of extrinsic stains in teeth usually goes through several stages: first, protein in saliva forms an acquired film on the surface of teeth, which provides a substrate for stain attachment; subsequently, the stain molecules bind to the tooth surface by electrostatic interaction, hydrogen bonding and calcium ion bridging. In addition, the micropore structure and roughness of enamel surface further promote stain deposition and restaining. Therefore, tooth discoloration is a complex process involving organic film formation, inorganic bonding and surface structure change.

In the prior art, tooth whitening products mainly adopt a single mechanism such as mechanical friction, chemical chelation, enzymatic hydrolysis to remove stains, physical adsorption or peroxide bleaching, but each of them has defects. In recent years, some literatures have begun to try to combine various mechanisms, such as:

China patent CN112043622A discloses a repairing whitening toothpaste including enzyme preparation and bioglass, which contains bromelain, papain, lysozyme and bioactive glass, and claims that whitening is capable of being achieved through the triple mechanisms of disintegrating stain attachments, inhibiting the regeneration of attachments and promoting the remineralization of enamel. However, this technology lacks the chelating desorption mechanism of the firmly bound stains, does not contain porous adsorption materials, and the sequential release of each component is not designed. When the enzyme acts with the repairing material at the same time, the action of the enzyme may be affected by the physical barrier of the repairing material, and the synergistic efficiency is limited.

International patent application US20180289606A1 discloses a multi-layer film delivery system, which realizes the step-by-step action of whitening agent and remineralization agent through the first soluble film and the second film. Although this technology is similar to this application in the idea of “staged action”, the core whitening agent is still peroxide bleaching agent, which may cause dentine hypersensitivity and oral irritation. And the release sequence is controlled by the physical dissolution speed of multilayer films, and the preparation process is complex, and the application form is limited to film agents, which is not suitable for daily toothpaste or tooth powder products.

In addition, China patent CN105142596A discloses an oral composition containing protease and soluble calcium phosphate remineralizer, and relates to a double-layer tablet scheme (rapid disintegration layer+slow erosion layer) to realize staged release. However, the staged release of this technology depends on the difference of disintegration and erosion speed of tablets, and is limited to tablet dosage forms; the oral composition also does not contain chelating desorption system and porous adsorption materials, and has insufficient ability to remove stains that have been firmly combined by calcium ion bridging.

To sum up, although the prior art has realized the importance of multi-mechanism combination and staged action, it has not fully covered the complete synergistic chain of “enzymatic film degradation-chelating desorption-adsorption repair”, nor adopted the porous carrier loading strategy based on pore size difference to realize simple, stable and suitable sequential release of powder/paste systems. Meanwhile, most technologies still rely on peroxide bleaching or lack of attention to the enamel repairing function, which leads to limited whitening effect, easy restaining or safety risks. Therefore, it is of great technical significance and application value to develop a whitening composition which may comprehensively aim at the mechanism of stain formation, achieve efficient stain removal through staged synergistic action and have the enamel repairing function.

### SUMMARY

The disclosure aims to overcome the shortcomings of the prior art and provide a tooth whitening composition with staged synergistic release function and a preparation method thereof. The composition achieves high-efficiency, mild and lasting whitening effect through the synergistic effect of enzymatic film degradation, chelating desorption and adsorption repair.

In order to achieve the above objectives, the present disclosure provides the following technical scheme.

A tooth whitening composition with staged synergistic release includes the following components in percentage by weight:
0.5%-2% of enzymatic film degradation component, 0.5%-5% of chelating desorption component, 5%-10% of porous adsorption material, 5%-10% of enamel repairing material, 45%-65% of abrasive base, 5%-10% of first porous carrier, 5%-10% of second porous carrier, 3%-5% of surfactant, 5%-10% of sweetener, and 0.5%-2% of a flavor.

The enzymatic film degradation component is loaded on the first porous carrier; the chelating desorption component is loaded on the second porous carrier. The porous adsorption material and the enamel repairing material are non-loaded particles and continuously exist in the system during use.

Further, the first porous carrier has a specific surface area of 30-150 m²/g and a pore volume of 0.8-1.5 cm³/g; and the second porous carrier has a specific surface area of 600-1000 m²/g and a pore volume of 0.5-1.0 cm³/g.

Further, the enzymatic film degradation component is selected from one or more of papain, bromelain and lysozyme, and the enzyme activity retention rate is more than or equal to 85%.

Further, the chelating desorption component is selected from one or more of sodium pyrophosphate, sodium tripolyphosphate, sodium hexametaphosphate and ethylenediaminetetraacetic acid disodium salt (ethylenediaminetetraacetic acid disodium salt).

Further, the porous adsorption material is activated carbon.

Further, the enamel repairing material is nano-hydroxyapatite.

Further, the first porous carrier and/or the second porous carrier are selected from silica or mesoporous silicon materials with different pore sizes.

Further, the abrasive base is one or more of calcium carbonate and calcium hydrogen phosphate.

Further, the surfactant is one or more of sodium lauroyl sarcosine and sodium cocoyl glutamate.

Further, the sweetener is one or more of xylitol and erythritol.

Further, the flavor is one or more of peppermint oil and cinnamon oil.

The disclosure also provides a preparation method of the tooth whitening composition, which includes the following steps:
step 1, carrier activation: activating the first porous carrier and the second porous carrier at 105 °C for 2 hours, and cooling to room temperature for later use;
step 2, low-temperature loading: dissolving the enzymatic film degradation component and the chelating desorption component with water respectively, soaking the enzymatic film degradation component in the first porous carrier for 2 hours at 25 °C, and soaking the chelating desorption component in the second porous carrier for 2 hours, then performing vacuum drying respectively, and passing through an 80-100 mesh sieve to obtain composite particle 1 and composite particle 2. The mass ratio of the enzymatic film degradation component to the first porous carrier is controlled between 1:2 and 1:20, and the mass ratio of the chelating desorption component to the second porous carrier is controlled between 1:2 and 1:20.
step 3, base material mixing: putting the abrasive base, the porous adsorption material and the enamel repairing material into a mixer for dry mixing for 15 minutes; and
step 4, final mixing: adding the composite particle 1, the composite particle 2, the surfactant, the sweetener and the flavor into a mixer, and mixing for 20 minutes until uniform; and
   (5) filling.

In addition, the disclosure also protects the use of the composition in preparing oral care products for removing extrinsic stains, reducing tooth restaining and repairing enamel.

The core technical principles of the disclosure are “staged synergistic release” and “enzymatic enhancement”.

### (1) Staged Release Mechanism

According to the disclosure, the porous carrier with obvious pore size difference is designed to realize the sequential release of each component:

The first stage (0-1 minute): the first porous carrier with larger pore size has a fast release rate, and the enzymatic film degradationcomponent is released rapidly (release rate≥90%), which decomposes the acquired pellicle on the tooth surface and exposes the calcium ion binding sites.

The second stage (2-3 minutes): the second porous carrier with smaller pore size slowly releases the chelating desorption component (release rate≥85%), efficiently chelates calcium ions, and cleaves the binding bond between stains and teeth.

The third stage (lasting after 2 minutes): with the progressive removal of the contaminated layer on the tooth surface, the porous adsorption material (activated carbon) and the enamel repairing material (nano-hydroxyapatite) continue to play a role in adsorbing free stains, repairing and polishing the enamel surface, reducing the roughness (Ra value can be reduced from 128 nm before treatment to 56 nm), thus reducing stain reattachment (restaining rate is reduced from 31% to 15%).

### (2) Enzymatic Enhancement

The acquired pellicle is an organic film covering the surface of enamel, with a thickness of about 0.1-1 micron, which is composed of glycoprotein and mucin in saliva. This film may not only be dyed by stain itself, but also physically block the contact between chelating agent and enamel surface. The papain or bromelain used in the disclosure specifically hydrolyzes the protein network in the acquired pellicle, decomposes the protein network into polypeptides and amino acids, and exposes the calcium ion binding sites on the surface of enamel, so that the chelating agent released later gives full play to the functions of efficiently chelating calcium ions and cleaving the stain binding bonds.

### (3) Protective Effect of Porous Carrier

According to the disclosure, the enzymatic component and the chelating component are respectively loaded in silica porous carriers with different pore diameters, so that not only the physical isolation and sequential release of the active components are realized, but also the deactivation caused by direct contact with friction agents, surfactants and the like is avoided. The results show that the enzyme activity retention rate in the carrier-loaded group is over 92%, while the enzyme activity retention rate in the direct mixing group is only 41.5%.

Through the cooperation of enzymolysis, chelation, adsorption and restoration, the disclosure realizes the efficient removal of extrinsic stains on teeth, low enamel damage and low restaining rate.

Compared with the prior art, the disclosure has the following beneficial effects.

### (1) Staged Synergistic Release Delivers Markedly Improved Whitening Efficiency

According to the disclosure, three-stage sequential release is realized through pore size difference, and the experimental results show that the stain removal rate ΔE value of Embodiments 1-3 reaches 12.0-13.0, which is significantly higher than that of Comparative example 5 (direct mixing group, ΔE=6.1), Comparative example 6 (enzyme-free group, ΔE=5.5) and Comparative example 9 (commercial product, ΔE=8.1), which proves the superiority of the multi-mechanism staged synergistic effect (P<0.05).

(2) The porous carrier protects the enzyme activity, and the product has excellent Stability.

The enzymatic component is loaded in porous carrier, which effectively avoids inactivation caused by direct contact with friction agent and surfactant. The accelerated aging experiment shows that the enzyme activity retention rate in the carrier-loaded group of the disclosure is 92.3±2.5%, while the enzyme activity retention rate in the direct mixing group is only 41.5±3.2%, and the difference is statistically significant (P<0.05).

(3) Mild Formula with High Biological Safety and No Damage to Enamel

According to the test, the relative dentin abrasion (RDA) values of Embodiments 1-3 of the present disclosure are only 50-55, which is far lower than that of commercial products (RDA=78) and standard tooth powder (RDA=100). After treatment, the enamel Vickers hardness (HV) was 342±10, showing no significant difference versus the pre-treatment value of 345±12, while the hardness of commercial products decreased to 301±11 after treatment. The score of the in vitro mucosal irritation test yields a grade 0 score, and none of the 30 test volunteers experience adverse reactions after four consecutive weeks of use.

### (4) Repairing Enamel Significantly Reduces the Restaining Rate

The nano-hydroxyapatite repairing material contained in the disclosure effectively fills the micropores on the enamel surface and reduces the surface roughness (the Ra value is reduced from 128±6 nm before treatment to 56±3 nm). The restaining experiment shows that the restaining rate of Embodiments 1-3 is only 13-16%, which is far lower than that of Comparative example 8 (31%) and commercial products (27%) without repairing materials.

### (5) Non-Peroxide Mechanism, No Stimulation Risk

No peroxide-based bleaching agents are incorporated into the present formulation, eliminating common side effects including tooth hypersensitivity and gingival irritation inherent to conventional peroxide whitening products and enabling safe daily oral care application.

### BRIEF DESCRIPTION OF THE DRAWINGS

FIG. 1 is a release kinetic curve of three components corresponding to Embodiment 1 of the present disclosure.

FIG. 2 is a release kinetic curve of Comparative example 1 of the present disclosure.

FIG. 3 is a release kinetic curve of Comparative example 2 of the present disclosure.

FIG. 4 is a release kinetic curve of Comparative example 3 of the present disclosure.

FIG. 5 is a release kinetic curve of Comparative example 4 of the present disclosure.

### DESCRIPTION OF EMBODIMENTS

The present disclosure will be further described with reference to embodiments and comparative examples. Unless otherwise specified, all contents are weight percent (wt %).

### Embodiments 1-3

According to the formula shown in Table 1, the tooth whitening compositions of Embodiments 1-3 were prepared according to the following preparation methods.

TABLE 1
Formula table of Embodiments 1-3

| | Name of raw | Embodiment | Embodiment | Embodiment |
|---|---|---|---|---|
| Components | material | 1 | 2 | 3 |
| Abrasive base | Calcium carbonate | 52.70% | 57.00% | 46.00% |
| First porous carrier | Macroporous silica | 10.00% | 8.00% | 5.00% |
| Second porous carrier | Microporous silica | 6.00% | 8.00% | 10.00% |
| Surfactant | Sodium lauroyl | 3.00% | 4.00% | 5.00% |
| | sarcosine | | | |
| Sweetener | Xylitol | 10.00% | 8.00% | 5.00% |
| Repairing material | Hydroxylapatite | 8.00% | 5.00% | 10.00% |
| Adsorption material | Activated carbon | 5.00% | 8.00% | 10.00% |
| Chelating desorption | Sodium | 3.00% | 0.50% | 5.00% |
| component | pyrophosphate | | | |
| Enzymatic film | Papain | 0.80% | 0.50% | 1.00% |
| degradation component | Bromelain | 0.50% | 0.50% | 1.00% |
| Flavor | Peppermint oil | 1.00% | 0.50% | 2.00% |

In Embodiments 1-3 of the present disclosure:

The first porous carrier is macroporous silica with an average pore diameter of about 75 nm, a specific surface area of 64 m²/g and a pore volume of 1.2 cm³/g; the second porous carrier is microporous silica with an average pore diameter of about 4 nm, a specific surface area of 800 m²/g and a pore volume of 0.8 cm³/g.

### Preparation Method

(1) Carrier activation: activating the first porous carrier and the second porous carrier at 105 °C for 2 hours, and cooling to room temperature for later use.
(2) Low-temperature loading: dissolving the enzymatic film degradation component and the chelating desorption component with water respectively, soaking the enzymatic film degradation component in the first porous carrier for 2 hours at 25 °C, and soaking the chelating desorption component in the second porous carrier for 2 hours, then performing vacuum drying respectively, and passing through an 80-100 mesh sieve to obtain composite particle 1 and composite particle 2.

In the specific design of the present disclosure, the mass ratio of the enzymatic film degradation component to the first porous carrier is controlled within the range of 1:2 to 1:20, and the mass ratio of the chelating desorption component to the second porous carrier is controlled within the range of 1:2 to 1:20. With the coordination of the above loading ratios, pore volumes and specific surface areas, the active substances are fully adsorbed into the internal pore channels of the porous carriers via capillary action, and barely exist in a free state on the material surface. This not only effectively prevents surfactants from impairing enzyme activity, but also guarantees a strictly controlled staged synergistic release effect when exposed to water in subsequent use.
(3) Base material mixing: putting the abrasive base, the porous adsorption material and the enamel repairing material into a mixer for dry mixing for 15 minutes;
(4) final mixing: adding the composite particle 1, the composite particle 2, the surfactant, the sweetener and the flavor into a mixer, and mixing for 20 minutes until uniform; and
(5) filling.
   Release Performance Test of Carriers with Different Pore Sizes

Taking Embodiment 1 as a comparative sample, the release performance of carriers with different pore sizes was tested through Comparative examples 1-4.

Comparative example 1: the pore diameters of the two carriers were the same (10-20 nm).

Comparative example 2: the pore diameters of the two carriers were similar (15-30 nm for the first carrier and 10-20 nm for the second carrier).

Comparative example 3: the pore configurations of carriers were opposite (the first carrier is 2-10 nm, and the second carrier is 20-80 nm).

Comparative example 4: both carriers had large pore sizes (100-150 nm).

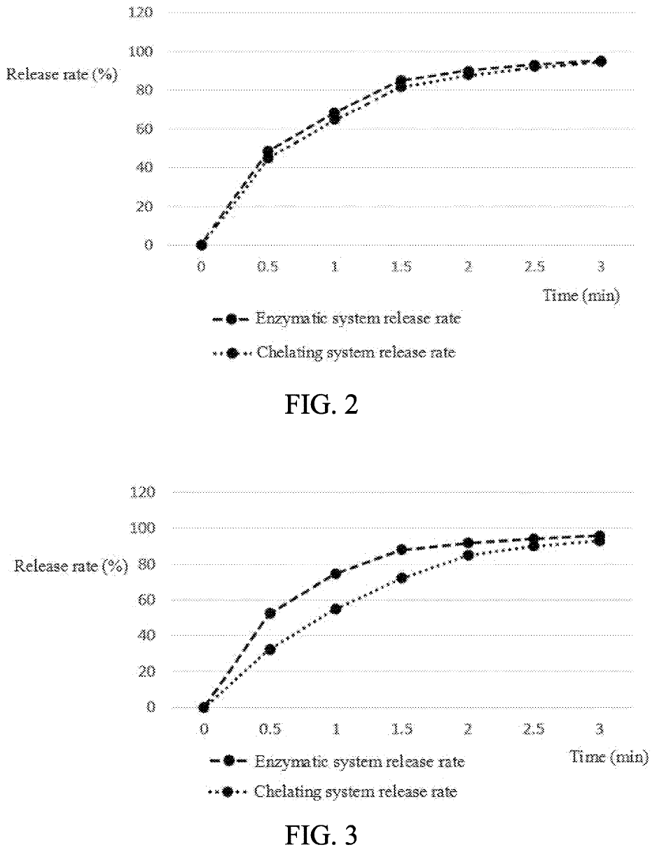

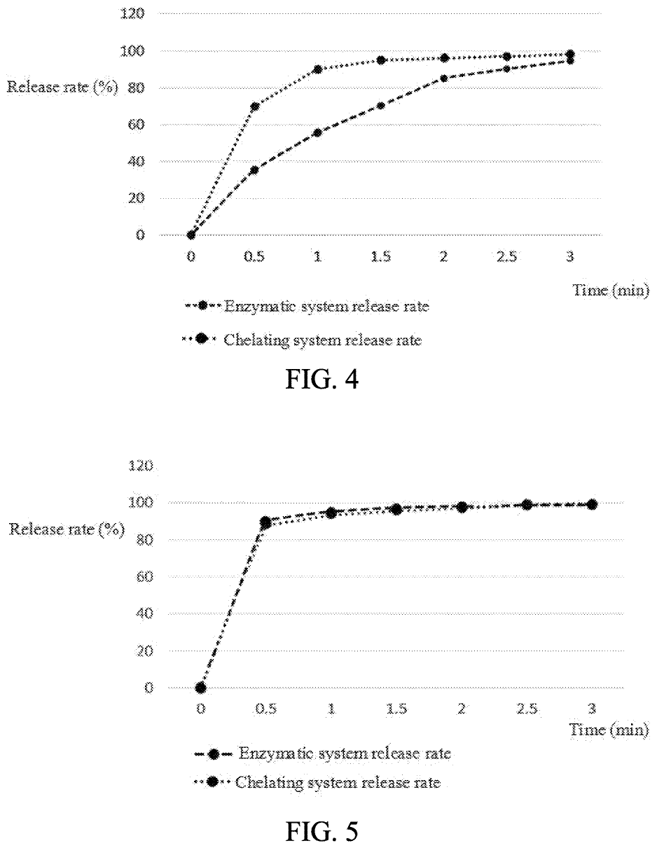

Test method: 1.0 g of the products of each comparative example were placed in simulated saliva at 37 °C and pH=6.8, and samples were taken at different time points, and the concentrations of papain and bromelain in the solution were determined by high performance liquid chromatography (HPLC). The concentration of pyrophosphate was determined by ion chromatography. The release rate-time curves of each component were plotted, as shown in FIG. 2-FIG. 5.

Results: obvious staged release behavior was achieved only when enzymes and chelating agents were separately loaded onto carriers (such as the scheme of the disclosure) with distinctly different pore sizes. The staged release phenomenon weakened or disappeared when the carriers possessed identical or marginally differentiated pore sizes.

### Performance Comparison of Different Formulations or Preparation Methods

Taking Embodiment 1 as a comparative sample, through Comparative examples 5-9, the performance of different formulations or preparation methods was compared:
Comparative example 5: compared with Embodiment 1, the enzyme and chelating agent were directly mixed without being loaded on the carriers.
Comparative example 6: compared with Embodiment 1, no enzymatic film degradation component was added.
Comparative example 7: compared with Embodiment 1, no chelating desorption component was added.
Comparative example 8: compared with Embodiment 1, no enamel repairing material was added.
Comparative example 9: common whitening tooth powder on the market.

### Performance Test and Results

1. Release kinetics test (Embodiment 1)

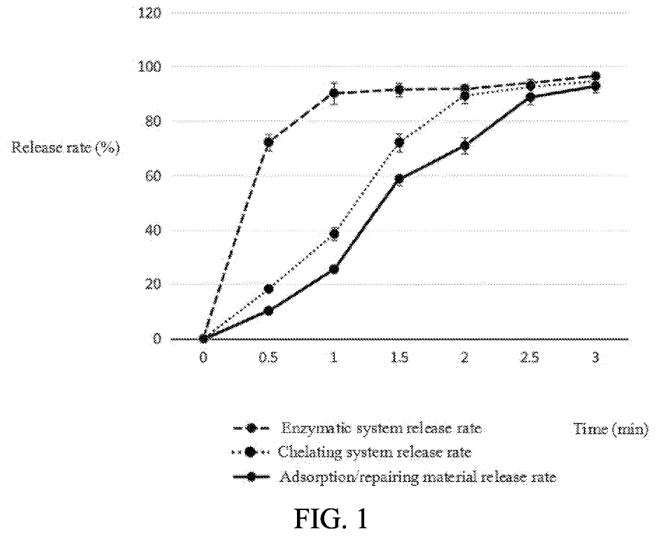

1.0 g of the product prepared in Embodiment 1 was placed into simulated saliva at 37 °C and pH-6.8. Samples were collected at 0, 0.5, 1, 1.5, 2, 2.5 and 3 minutes respectively. HPLC was adopted to determine the concentrations of papain and bromelain in the solution. Ion chromatography was used to measure the concentration of pyrophosphate. An ultraviolet spectrophotometry method was applied to detect the turbidity change of hydroxyapatite, and the release rate-time curves of each component were plotted, as shown in FIG. 1.

Results: the release rate of enzymatic components reached 90% within 0-1 minute, and the release rate of chelating components reached 85% during 1-2 minutes. The release rate of the repairing material achieved the maximum release rate after 2 minutes, which verifies the three-stage release mechanism. It was noted that the porous adsorption material and enamel repairing material (nano-hydroxyapatite) existed as unloaded free particles in the system. The observed peak release rate the repairing material after 2 minutes essentially corresponded to the maximum rate of effective contact, dispersion, adsorption and remineralization reactions at the enamel interface. During the initial two minutes, extrinsic organic acquired pellicle and deeply bound stains formed a dense physical barrier on the tooth surface and prevented free repair particles from contacting and depositing inside enamel micropores. After 2 minutes, the previously loaded enzymatic and chelating components thoroughly hydrolyzed the organic fouling layer and removed bound stains via chelation in a strict sequential order. Micropores on the enamel surface became fully exposed, which induced extensive interfacial interaction of free nano-hydroxyapatite within a short timeframe; the particles rapidly precipitated and filled the micropores. The objective test data reflected an ideal staged synergistic repairing effect characterized by the peaked release rate of the repairing material after 2 minutes.

2. Test of enzyme activity retention rate

| | Enzyme activity retention rate (%, |
|---|---|
| Samples | average value ± standard deviation) |
| Embodiment 1 (load | 92.3 ± 2.5 |
| carrier group) | |
| Comparative example 5 | 41.5 ± 3.2 |
| (direct mixing group) | |

Conclusion: Porous carrier is capable of effectively protecting the enzyme activity, and the difference is statistically significant (P < 0.05).

3. Testing enamel surface roughness and
relative dentin abrasion (RDA) value

| | Ra (nm, average | RDA value (average |
|---|---|---|
| | value ± standard | value ± standard |
| Samples | deviation) | deviation) |
| Before treatment | 128 ± 6 | 90 ± 3 |
| Embodiment 1 | 56 ± 3 | 50 ± 2 |
| Embodiment 2 | 60 ± 3 | 55 ± 2 |
| Embodiment 3 | 58 ± 3 | 53 ± 2 |
| Comparative example | 119 ± 5 | 85 ± 3 |
| 8 (No repairing | | |
| material) | | |
| Comparative example | 95 ± 4 | 78 ± 3 |
| 9 (Commercial | | |
| product) | | |

Conclusion: the embodiments containing hydroxyapatite are capable of significantly reducing the surface roughness of enamel, and the RDA value is much lower than that of the commercial products, with better gentleness (P < 0.05).

4. Tooth enamel Vickers hardness test

| | Hardness (HV, average value ± standard |
|---|---|
| Samples | deviation) |
| Before treatment | 345 ± 12 |
| After treatment of | 342 ± 10 |
| Embodiment 1 | |
| Comparative example 9 | 301 ± 11 |
| (Commercial products) | |

Conclusion: the disclosure has no obvious damage to enamel, and the safety is significantly better than that of the commercial products (P < 0.05).

5. Stain removal rate test (ΔE value)

| | ΔE value (average | |
|---|---|---|
| | value ± standard | Effect |
| Samples | deviation) | evaluation |
| Embodiment 1 | 12.5 ± 0.6 | Excellent |
| Embodiment 2 | 13.0 ± 0.5 | Excellent |
| Embodiment 3 | 12.0 ± 0.7 | Excellent |
| Comparative example | 6.1 ± 0.4 | Poor |
| 5 (Direct mixing) | | |
| Comparative example | 5.5 ± 0.3 | Poor |
| 6 (Enzyme-free) | | |
| Comparative example | 7.6 ± 0.4 | Average |
| 7 (No chelation) | | |
| Comparative example | 9.2 ± 0.5 | Good |
| 8 (No repair treatment) | | |
| Comparative example | 8.1 ± 0.4 | Good |
| 9 (Commercial | | |
| product) | | |

Conclusion: Embodiments 1-3 showed the highest ΔE value, indicating that the synergistic effect of the system significantly improved the tooth whitening effect (P < 0.05).

6. Restaining test

| | Restaining rate (%, average value ± |
|---|---|
| Samples | standard deviation) |
| Embodiment 1 | 15 ± 1.2 |
| Embodiment 2 | 13 ± 1.0 |
| Embodiment 3 | 16 ± 1.3 |
| Comparative example 5 | 29 ± 2.1 |
| (Direct mixing) | |
| Comparative example 6 | 32 ± 2.3 |
| (Enzyme-free) | |
| Comparative example 7 | 35 ± 2.5 |
| (No chelation) | |
| Comparative example 8 | 31 ± 2.2 |
| (No repair treatment) | |
| Comparative example 9 | 27 ± 2.0 |
| (Commercial product) | |

Conclusion: the embodiments containing repairing materials are capable of significantly reducing the restaining rate, indicating that the repairing effect of hydroxyapatite effectively reduces the re-deposition of the stain (P < 0.05).

### 7. Oral Safety Test

In vitro mucosal irritation test: the irritation score of all the samples in the embodiments is grade 0 (no irritation).

Human trial experiment: 30 volunteers continued to use the sample of the embodiments for 4 weeks, and no adverse reactions such as oral mucosal irritation and gingival sensitivity occurred.

To sum up, the tooth whitening composition with staged synergistic release provided by the disclosure is obviously superior to the prior art in terms of stain removal rate, restaining inhibition and enamel restoration, and has good safety, significant technical progress and application value.

What is claimed is:

1. A tooth whitening composition with staged synergistic release, comprising following components in percentage by weight:
0.5%-2% of enzymatic film degradation component, 0.5%-5% of chelating desorption component, 5%-10% of porous adsorption material, 5%-10% of enamel repairing material, 45%-65% of abrasive base, 5%-10% of first porous carrier, 5%-10% of second porous carrier, 3%-5% of surfactant, 5%-10% of sweetener, and 0.5%-2% of a flavor;
a pore size of the first porous carrier is 50-100 nm, and a pore size of the second porous carrier is 2-20 nm;
the enzymatic film degradation component is loaded on the first porous carrier; the chelating desorption component is loaded on the second porous carrier; and
the composition realizes staged sequential release through a pore size difference between the first porous carrier and the second porous carrier.

2. The composition according to claim 1, wherein the enzymatic film degradation component is selected from one or more of papain, bromelain and lysozyme.

3. The composition according to claim 1, wherein the chelating desorption component is selected from one or more of sodium pyrophosphate, sodium tripolyphosphate, sodium hexametaphosphate and ethylenediaminetetraacetic acid disodium salt.

4. The composition according to claim 1, wherein the porous adsorption material is activated carbon; and/or the enamel repairing material is nano-hydroxyapatite.

5. The composition according to claim 1, wherein the first porous carrier and/or the second porous carrier are selected from silica or mesoporous silicon materials with different pore sizes; wherein the first porous carrier has a specific surface area of 30-150 m²/g and a pore volume of 0.8-1.5 cm³/g; and the second porous carrier has a specific surface area of 600-1000 m²/g and a pore volume of 0.5-1.0 cm³/g.

6. The composition according to claim 1, wherein the abrasive base is one or more of calcium carbonate and calcium hydrogen phosphate; the surfactant is one or more of sodium lauroyl sarcosine and sodium cocoyl glutamate; the sweetener is one or more of xylitol and erythritol; and the flavor is one or more of peppermint oil and cinnamon oil.

7. The composition according to claim 1, wherein the composition is tooth powder.

8. A preparation method for the tooth whitening composition according to claim 1, comprising following steps:
step 1, carrier activation: activating the first porous carrier and the second porous carrier at 105 °C for 2 hours, and cooling to room temperature for later use;
step 2, low-temperature loading: dissolving the enzymatic film degradation component and the chelating desorption component with water respectively, soaking the enzymatic film degradation component in the first porous carrier for 2 hours at 25 °C, and soaking the chelating desorption component in the second porous carrier for 2 hours, then performing vacuum drying respectively, and passing through an 80-100 mesh sieve to obtain composite particle 1 and composite particle 2;
step 3, base material mixing: putting the abrasive base, the porous adsorption material and the enamel repairing material into a mixer for dry mixing for 15 minutes;
step 4, final mixing: adding the composite particle 1, the composite particle 2, the surfactant, the sweetener and the flavor into a mixer, and mixing for 20 minutes until uniform; and
step 5, filling.

9. The preparation method according to claim 8, wherein in step 2, a mass ratio of the enzymatic film degradation component to the first porous carrier is 1:2 to 1:20; a mass ratio of the chelating desorption component to the second porous carrier is 1:2 to 1:20.
